(19) United States
(12) Patent Application Publication
(10) Pub. No.: US 2025/0085265 A1
(43) Pub. Date: Mar. 13, 2025

(54) Pharmaceutical Apparatus And Method Of Manufacturing A Patient-Tailored Pharmaceutical Tablet

(71) Applicant: Cyrus Scientific, LLC, Lexington, KY (US)

(72) Inventors: Amin ABEDINI, Lexington, KY (US); Matthew Kyle DEFRESE, Lexington, KY (US)

(73) Assignee: Cyrus Scientific, LLC, Lexington, KY (US)

(21) Appl. No.: 18/956,742

(22) Filed: Nov. 22, 2024

Related U.S. Application Data
(63) Continuation of application No. PCT/US2023/023955, filed on May 31, 2023.
(60) Provisional application No. 63/347,797, filed on Jun. 1, 2022.

(51) Int. Cl. G01N 33/15; A61J 3/10; B30B 11/00; B30B 11/04; B30B 15/30

(52) U.S. Cl. CPC G01N 33/15; A61J 3/10; B30B 11/005; B30B 11/04; B30B 15/302; A61J 2200/70; A61J 2200/74; A61J 2205/30

(57) ABSTRACT

A pharmaceutical apparatus is provided. In another aspect, a pharmaceutical apparatus includes a tablet press configured to compress a first powdered ingredient and a second powdered ingredient into a multi-layer pharmaceutical tablet. A further aspect employs an analytical and quality control table including at least one of a process analytical technologies (“PAT”) sensor and an imaging device. In another aspect, the analytical and quality control table further includes a weighing device configured to operate with a standard deviation between 0 and 3. In yet another aspect, the pharmaceutical apparatus utilizes a programmable controller to operate the pharmaceutical apparatus.

### CROSS-REFERENCE TO RELATED APPLICATIONS

The present application is a continuation of PCT Application Serial No. PCT/US2023/023955, filed May 31, 2023, which claims priority to U.S. Provisional Application Ser. No. 63/347,797, filed on Jun. 1, 2022, both of which are incorporated by reference herein.

### BACKGROUND AND SUMMARY

The present disclosure relates to an apparatus and method for manufacturing patient-tailored pharmaceutical tablets.

Personalized medicines are a principal objective in drug development. Precision dosing can provide superior control of target drug blood concentrations which is expected to optimize patient outcomes by maximizing drug efficacy and minimizing risk of adverse effects. Existing technologies for personalized medicines are generally limited to diagnostic tools used by medical professionals to select the type and amount of medication for an individual, but that flexibility often does not extend to the prescribed drug product. Patient-tailored dosage control of pharmaceutical tablets does not exist at scales suitable for commercial manufacturing. As such, patients often require a number of tablets and/or tablet splitting which results in poor patient compliance, drug performance, and dosage control.

Current approaches to precision dosing of pharmaceutical tablets typically utilize variations of 3D-printing technologies. While there is great promise for 3D-printing to deliver personalized medicines, there are also great difficulties in addressing inherent technical challenges and limitations. The three main 3D-printing techniques for pharmaceuticals include fused deposition modeling (FDM), powder bed fusion (PBF), and stereolithography (SLA), each of which are incompatible with many drugs due to excess heat and/or UV exposure. In addition, the effects of these processing techniques on drug product performance are poorly understood and rely on excipient materials, such as UV-cured resins, that do not have a robust history of use in drug products—all of which brings concerns of feasibility, quality, safety, and reliability. Thus, there is a desire for an alternative method of producing personalized pharmaceutical tablets.

In accordance with the present invention, a pharmaceutical apparatus is provided. In another aspect, a pharmaceutical apparatus includes a tablet press configured to compress a first powdered ingredient and a second powdered ingredient into a multi-layer pharmaceutical tablet. A further aspect employs an analytical and quality control table including at least one of a process analytical technologies (“PAT”) sensor and at least one 2D and/or 3D imaging device, such as a camera and/or a 3D scanner. In yet another aspect, the pharmaceutical apparatus utilizes a programmable control unit to operate the pharmaceutical apparatus. In another aspect, the analytical and quality control table employs at least one weighing device configured to operate with a standard deviation between 0 and 3.

A further aspect provides a method of manufacturing pharmaceutical tablets. In another aspect, a user provides details of a pharmaceutical tablet to a programmable controller and the programmable controller directs a tablet press to compress a first and second powdered ingredients into a pharmaceutical tablet before the pharmaceutical tablet is transferred to an analytical and quality control table for inspection. A further aspect includes a user-defined customization of a first and second pharmaceutical tablet, such that sizes of the first and second pharmaceutical tablets are equivalent, while the sizes of individual layers by ingredient may vary independently within the tablets.

The present pharmaceutical apparatus and multi-layer pharmaceutical tablet manufacturing method is advantageous over prior devices and methods. For example, the present pharmaceutical apparatus can allow customization of pharmaceutical tablets tailored to the needs of individual patients without exposure to the risks of 3D printing. This customization may allow for varying dosages of one or more active pharmaceutical ingredient without changing the dimensions of the pharmaceutical tablet, thus allowing a patient to maintain familiarity and visual verification of a dose with a prescribed medication. The label or mark incorporated on each tablet further allows for traceability and/or verification of the dosage of each manufactured prescription and/or individual tablet. Additionally, the pharmaceutical apparatus is not limited to pharmaceutical tablets containing a specific drug; therefore, a number of pharmaceutical products may be used as an active pharmaceutical ingredient in such a multi-layered pharmaceutical tablet or “polypill,” with independent dose adjustment of each active component according to patient-specific needs. These active pharmaceutical ingredients include, but are not limited to, active components of Warfarin, Lofexidine, Metformin, Janumet, and Prednisone.

In general, patient-tailored precision dosing is particularly well suited to the production of pharmaceutical drug products and active pharmaceutical ingredients with narrow therapeutic indices. Additionally, pharmaceutical drug products and active pharmaceutical ingredients prescribed to patient populations with a wide range of dosing levels due to factors such as weight, sex, age, disease indication, metabolic function, genetics, titration prescribing requirements, patient sensitivity to adverse effects, and comorbidities would benefit from such patient-tailored precision dosing.

Furthermore, in-line measurement and inspection of the pharmaceutical tablet provides immediate quality control to ensure the dosage, accuracy, efficacy, and safety of the pharmaceutical tablet. Finally, the use of a programmable controller allows the automated manufacture of the pharmaceutical tablets with minimal manpower requirements. Additional advantages and features will be disclosed in the following description and appended claims as well as in the accompanying drawings.

### DRAWINGS

The drawings described herein are for illustrative purposes only of selected embodiments and not all possible implementations, and are not intended to limit the scope of the present disclosure.

FIG. 1 is a perspective view of the present pharmaceutical apparatus;

FIG. 2 is a side elevational view of the pharmaceutical apparatus of FIG. 1;

FIG. 3 is a top elevational view of the pharmaceutical apparatus of FIG. 1;

FIG. 4 is a side elevational view of the pharmaceutical apparatus of FIG. 1;

FIG. 5 is a side elevational view of an exemplary pharmaceutical tablet manufactured by the pharmaceutical apparatus of FIGS. 1-4;

FIG. 6 is a top elevational view of the compression table and the first and second dosing units of the pharmaceutical apparatus of FIGS. 1-4;

FIG. 7 is a bottom elevational view of the first and second dosing units of the pharmaceutical apparatus of FIGS. 1-4;

FIG. 8 is a diagrammatic view showing a process of measuring and inspecting a pharmaceutical tablet on an analytical and quality control table;

FIG. 9 is a schematic representation of the controller of the pharmaceutical apparatus of FIGS. 1-4;

FIG. 10 is a side elevational view of the tablet press of the pharmaceutical apparatus of FIGS. 1-4 during operation of the pharmaceutical apparatus;

FIG. 11 is a cross-sectional view, taken along line 11-11 of FIG. 10, of the tablet press;

FIG. 12 is a side elevational view of the tablet press of the pharmaceutical apparatus of FIGS. 1-4 during operation of the pharmaceutical apparatus;

FIG. 13 is a cross-sectional view, taken along line 13-13 of FIG. 12, of the tablet press;

FIG. 14A is a first portion of a flowchart illustrating a calibration method of the pharmaceutical apparatus of FIGS. 1-4;

FIG. 14B is a second portion of the flowchart illustrating a calibration method of the pharmaceutical apparatus of FIGS. 1-4; and

FIG. 15 is a flowchart illustrating an operation method of the pharmaceutical apparatus of FIGS. 1-4.

Corresponding reference numerals indicate corresponding parts throughout the several views of the drawings.

### DETAILED DESCRIPTION

Example embodiments will now be described more fully with reference to the accompanying drawings.

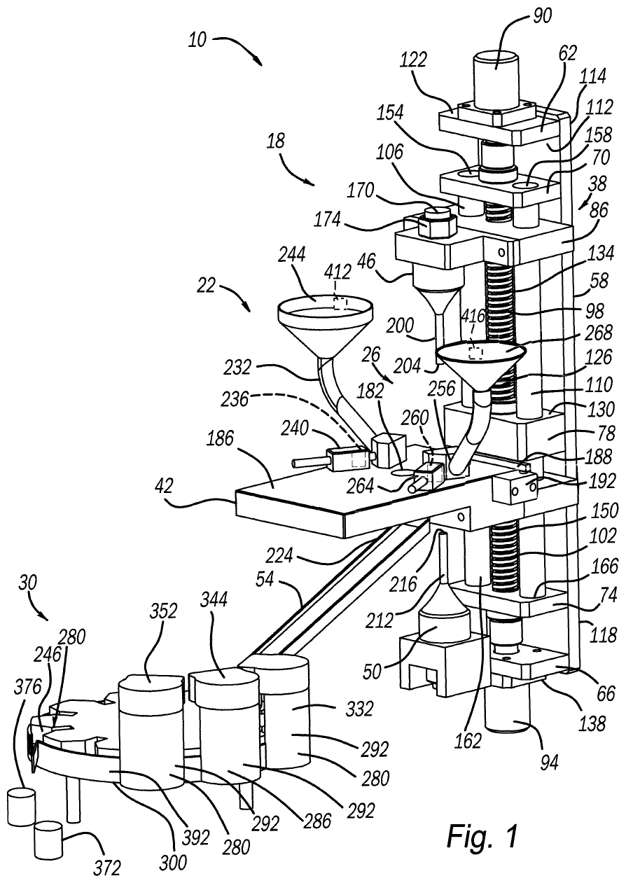

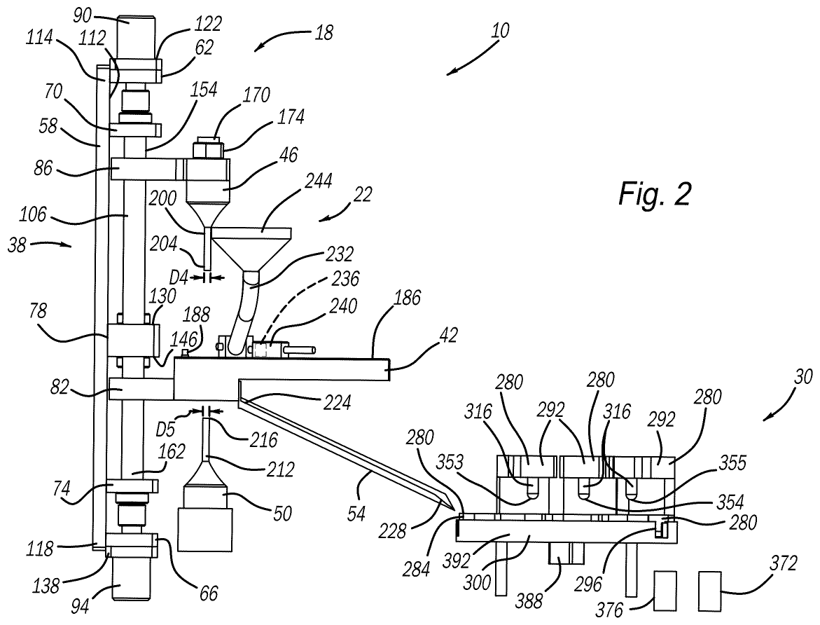

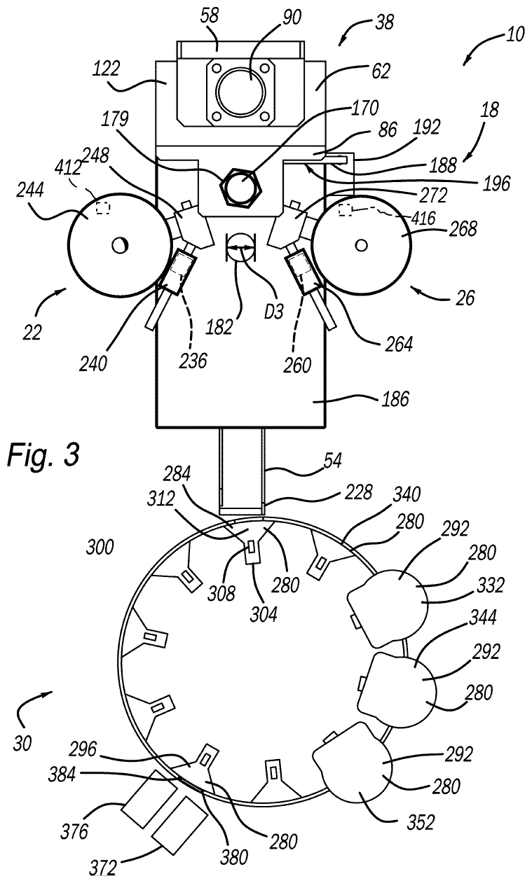

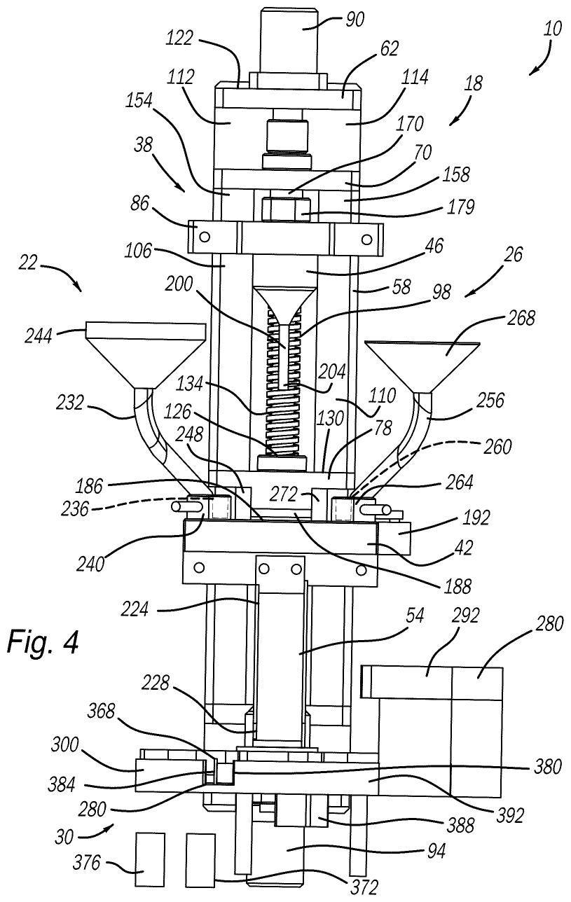

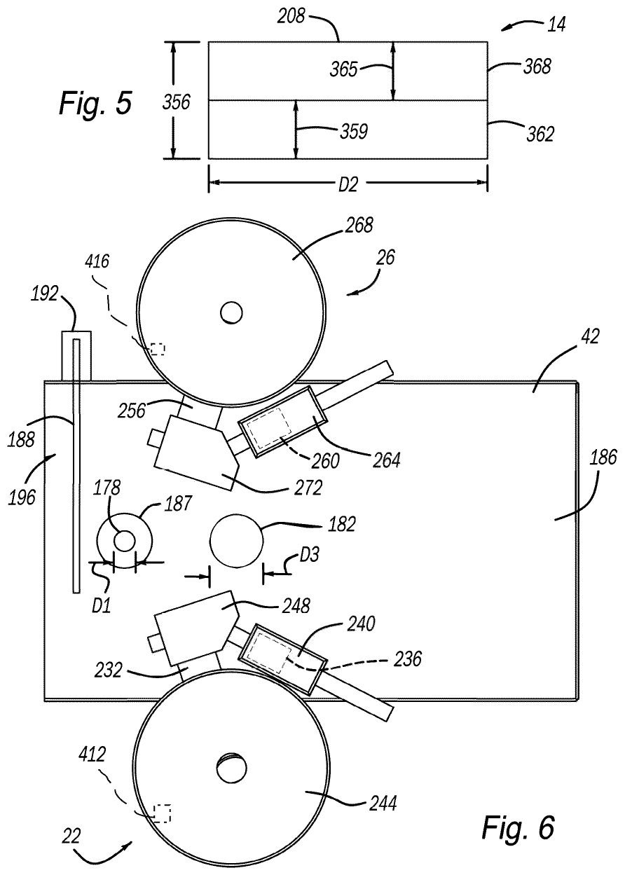

FIGS. 1-4 provide a first embodiment of a pharmaceutical apparatus 10, which is configured to manufacture a multi-layer pharmaceutical tablet 14, such as pharmaceutical tablet 14 shown in FIG. 5. Although pharmaceutical tablet 14 includes two layers, the pharmaceutical apparatus may manufacture pharmaceutical tablets including more layers. Pharmaceutical apparatus 10 includes a tablet press 18, a first dosing unit 22, a second dosing unit 26, an analytical and quality control table or analytical and quality control platform (hereinafter “A&QC unit”) 30, and a controller 34.

Tablet press 18 includes a frame 38, a compression table 42, an upper punch 46, a lower punch 50, and a discharge chute 54. Frame 38 of tablet press 18 includes a backplate 58, a top mounting plate 62, a bottom mounting plate 66, an upper guidepost plate 70, a lower guidepost plate 74, a support plate 78, a compression table mount-plate 82, and an upper punch plate 86. Frame 38 also includes a first motor 90, a second motor 94, a first ball screw 98, a second ball screw 102, a first guidepost 106, and a second guidepost 110. Backplate 58 is substantially flat and extends in a vertical direction with top and bottom mounting plates 62, 66, upper and lower guidepost plates 70, 74, and support plate 78 extending horizontally from a first face 112 of backplate 58. In some configurations, backplate 58 may be fixed to a wall, table, or other suitable base. Top mounting plate 62 extends from a top portion 114 of backplate 58 and bottom mounting plate 66 extends from a bottom portion 118 of backplate 58. Furthermore, top mounting plate 62 includes an aperture (not shown) and bottom mounting plate 66 includes an aperture (not shown). In some configurations, bottom mounting plate 66 may be fixed to a table, the floor or other suitable base.

First motor 90 is fixed to an upper surface 122 of top mounting plate 62 by bolts or another suitable fastener. First motor 90 is an electromagnetic actuator that may be a stepper motor, a servo motor, or any other suitable motor and associated transmission capable of rotational drive. Operation of first motor 90 is controlled by controller 34. First motor 90 is positioned above the aperture of top mounting plate 62. A first end (not shown) of first ball screw 98 extends through the aperture of top mounting plate 62 and is coupled to first motor 90, such that first motor 90 is configured to rotate first ball screw 98. First ball screw 98 extends from top mounting plate 62 to support plate 78. A second end 126 of first ball screw 98 is rotatably coupled to an upper surface 130 of support plate 78. First ball screw 98 includes a threaded exterior 134 extending a length of first ball screw 98.

Similarly, second motor 94 is fixed to a lower surface 138 of bottom mounting plate 66 by bolts or another suitable fastener. Second motor 94 is an electromagnetic actuator that may be a stepper motor, a servo motor, or any other suitable motor and associated transmission capable of rotational drive. Operation of second motor 94 is controlled by controller 34. Second motor 94 is positioned below the aperture of bottom mounting plate 66. Moreover, a first end (not shown) of second ball screw 102 extends through the aperture of bottom mounting plate 66 and is coupled to second motor 94, such that second motor 94 is configured to rotate second ball screw 102. Second ball screw 102 extends from the aperture of bottom mounting plate 66 to support plate 78. A second end 142 of second ball screw 102 is rotatably coupled to a lower surface 146 of support plate 78. Second ball screw 102 includes a threaded exterior 150 extending a length of second ball screw 102.

Upper guidepost plate 70 is located between top mounting plate 62 and support plate 78. Upper guidepost plate 70 includes a first aperture (not shown) configured to allow first ball screw 98 to extend therethrough. A first end 154 of first guidepost 106 is fixed to upper guidepost plate 70. Similarly, a first end 158 of second guidepost 110 is fixed to upper guidepost plate 70.

Lower guidepost plate 74 is located between bottom mounting plate 66 and support plate 78. Lower guidepost plate 74 includes a first aperture (not shown) configured to allow second ball screw 102 to extend therethrough. A second end 162 of first guidepost 106 is fixed to lower guidepost plate 74. Similarly, a second end 166 of second guidepost 110 is fixed to lower guidepost plate 74.

Support plate 78 includes a first aperture (not shown) configured to allow first guidepost 106 to extend therethrough. Support plate 78 also includes a second aperture (not shown) configured to allow second guidepost 110 to extend therethrough.

Compression table mount-plate 82 includes a nut mechanism (e.g., a nut ball) (not shown) configured to slidably and non-rotatably receive second ball screw 102, such that compression table mount-plate 82 traverses second ball screw 102 in the vertical direction when second ball screw 102 is rotated. Compression table mount-plate 82 is positioned along second ball screw 102 between support plate 78 and bottom mounting plate 66. Compression table mount-plate 82 includes a first aperture (not shown) configured to allow first guidepost 106 to extend therethrough. Compression table mount-plate 82 also includes a second aperture (not shown) configured to allow second guidepost 110 to extend therethrough. Compression table mount-plate 82 is configured to allow compression table 42 to be coupled thereto by a weld, threaded fasteners, or another suitable fastening method. In some configurations, compression table 42 may be removably coupled to compression table mount-plate 82 such that compression plate 42 may be easily interchanged. Compression table 42 may be partially atop compression table mount-plate 82. In some configurations, compression table 42 may extend horizontally from compression table mount-plate 82.

Upper punch plate 86 includes a nut mechanism (e.g., a nut ball) (not shown) configured to slidably and non-rotatably receive first ball screw 98, such that upper punch plate 86 traverses first ball screw 98 in the vertical direction when first ball screw 98 is rotated. Upper punch plate 86 is positioned along first ball screw 98 between support plate 78 and top mounting plate 62. Upper punch plate 86 includes a first aperture (not shown) configured to allow first guidepost 106 to extend therethrough. Upper punch plate 86 also includes a second aperture (not shown) configured to allow second guidepost 110 to extend therethrough. Upper punch plate 86 is configured to allow upper punch 46 to be fixed thereto. Upper punch 46 may be fixed to upper punch plate 86 by a bolt 170 and a nut 174, or another suitable fastening method.

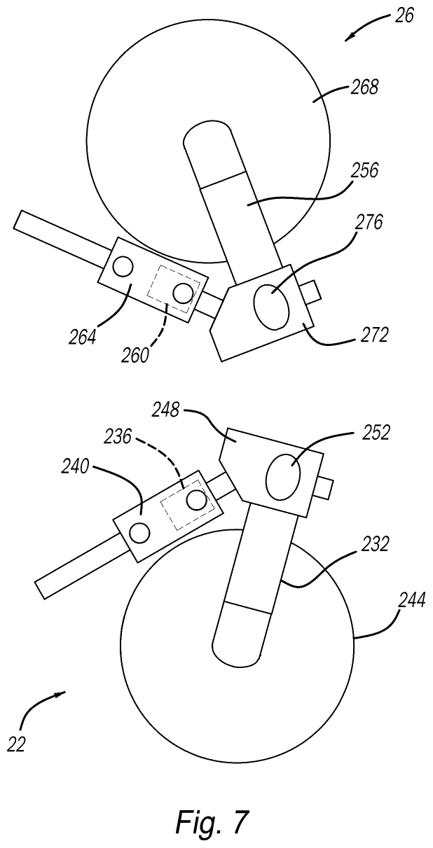

Compression table 42, shown in FIG. 6, includes a die 178, a first aperture 182, and a substantially flat upper surface 186. Die 178 may be part of a removable die insert 187 which can be inserted into a second aperture (not shown) of compression table 42, thus allowing the manufacture of varying sized and shaped pharmaceutical tablets. Die insert 187 may be coupled to compression table 42, by a set screw, interference fit, or another suitable fastening method. Compression table 42 travels with compression table mount-plate 82 when compression table mount-plate 82 traverses second ball screw 102 in the vertical direction when second ball screw 102 rotates. Die 178 of compression table 42 may be sized such that a diameter D1 of die 178 of compression table 42 is approximately the same diameter as an outer diameter D2 of pharmaceutical tablet 14. First aperture 182 of compression table 42 is configured to include a larger diameter D3 than diameter D1 of die 178 of compression table 42.

A wiper 188 is configured to rotate across upper surface 186 of compression table 42. Wiper 188 is rotatably coupled to a third motor 192, which is configured to rotate wiper 188. Operation of third motor 192 is controlled by controller 34. Wiper 188 is configured to extend above die 178 of compression table 42 when wiper 188 is rotated. Wiper 188 is configured to transfer pharmaceutical tablet 14 from a position above die 178 to first aperture 182 of compression table 42 when wiper 188 rotates. Wiper 188 is configured to remain in a first position 196 adjacent to die 178 of compression table 42 opposite first aperture 182 of compression table 42.

Returning to FIGS. 1-4, upper punch 46 includes an upper compressing rod 200 extending downward from upper punch plate 86. upper compressing rod 200 is concentric with die 178 of compression table 42. Upper compressing rod 200 is advanced into die 178 of compression table 42 as first ball screw 98 is rotated in such a way that upper punch plate 86 is driven downward. Upper compressing rod 200 is withdrawn from die 178 of compression table 42 as first ball screw 98 is rotated in such a way that upper punch plate 86 is driven upward. A first end 204 of upper compressing rod 200 includes an outer diameter D4, which may be approximately equal to diameter D1 of die 178 of compression table 42. In addition, first end 204 of upper compressing rod 200 is configured to press an upper surface 208 of pharmaceutical tablet 14 into a desired shape. In some configurations, upper compressing rod 200 may be removable from upper punch 46 such that upper compressing rod 200 may be easily interchanged.

Lower punch 50 remains in a fixed location. Lower punch 50 may be fixed to a table or other platform. Lower punch 50 includes a lower compressing rod 212 extending upward. Lower compressing rod 212 is concentric with die 178 of compression table 42. When compression table 42 is moved downward in the vertical direction, lower compressing rod 212 is received in die 178 of compression table 42. When compression table 42 is moved upward in the vertical direction, lower compressing rod 212 is withdrawn from die 178 of compression table 42. A first end 216 of lower compressing rod 212 includes an outer diameter D5, which may be approximately equal to diameter D1 of die 178 of compression table 42. In addition, first end 216 of lower compressing rod 212 is configured to press a lower surface 220 of pharmaceutical tablet 14 into a desired shape. In some configurations, lower compressing rod 212 may be removable from lower punch 50 such that lower compressing rod 212 may be easily interchanged.

Discharge chute 54 of tablet press 18 may be an elongated concave chute extending from compression table 42 to A&QC unit 30. Discharge chute 54 is configured such that a first end 224 of discharge chute 54 is located adjacent to compression table 42. A second end 228 of discharge chute 54 is located adjacent to A&QC unit 30. First end 224 of discharge chute 54 may be at a higher location than second end 228 of discharge chute 54. First end 224 of discharge chute 54 may be positioned below first aperture 182 of compression table 42. Discharge chute 54 is configured such that pharmaceutical tablet 14 falls to discharge chute 54 after passing through first aperture 182 of compression table 42. First end 224 of discharge chute 54 may be coupled to compression table 42 by bolts or another suitable fastener.

First dosing unit 22, shown in FIGS. 1-4, 6, and 7, includes a first conduit 232, a first vibration unit 236, and a first actuator 240. First dosing unit 22 is configured to be placed atop upper surface 186 of compression table 42. First dosing unit 22 guides a first powdered ingredient (not shown), which may be a first powdered or granulated active pharmaceutical ingredient, a first powdered or granulated inactive ingredient, a first powdered or granulated blend of active pharmaceutical ingredients, or a first powdered or granulated blend of active pharmaceutical ingredients and inactive ingredients. The first powdered ingredient is dispensed into an intake end 244 of first conduit 232. The first powdered ingredient may be dispensed by an automated hopper or a manually-controlled hopper, or may be manually dispensed by a user. Intake end 244 of first conduit 232 may be configured as a funnel.

In some embodiments, first dosing unit 22 may include a first pre-pressing sensor 412 configured to detect composition data of first powdered ingredient as first powdered ingredient flows through first dosing unit 22. Controller 34 receives the detected composition data from first pre-pressing sensor 412. Controller 34 then determines at least one of potency, water content, impurities, or a presence of polymorphs of first powdered ingredient from data collected by first pre-pressing sensor 412. First pre-pressing sensor 412 may be any satisfactory analytical technology, such as an ultraviolet-visible spectrometer, a near-infrared spectrometer, a Raman spectrometer, a fluorescence spectrometer, a refractometer, or a micro-indentation sensor. First pre-pressing sensor 412 is connected to and operably sends sensing signals to controller 34. First pre-pressing sensor 412 provides in-line identification and composition of raw material powders, which may be used to adjust the tablet press process to produce tablets/layers of accurate dose and composition, based on the data detected by first pre-pressing sensor 412. For example, the preferred

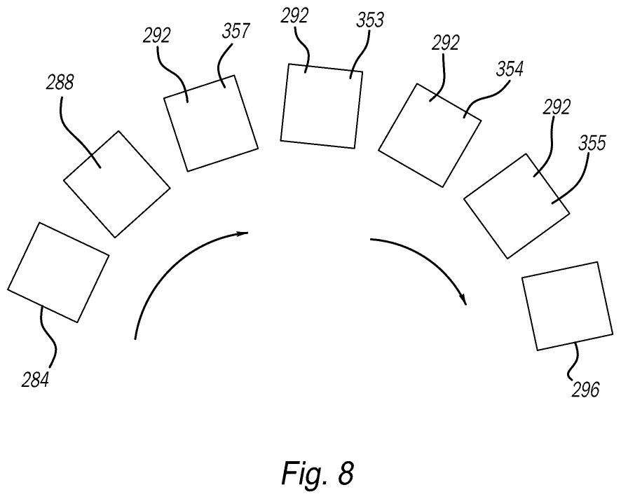

First conduit 232 is configured to allow the first powdered ingredient to flow through first conduit 232 from intake end 244 to a discharge end 248. Discharge end 248 of first conduit 232 includes an opening 252 (shown in FIG. 7) that is configured to allow the first powdered ingredient to flow into die 178 of compression table 42 when discharge end 248 is placed above die 178. In addition, discharge end 248 of first conduit 232 is configured such that the first powdered ingredient is unable to pass between discharge end 248 of first conduit 232 and upper surface 186 of compression table 42 when discharge end 248 of first conduit 232 is in contact with upper surface 186 of compression table 42, thus preventing the first powdered ingredient from flowing out of first conduit 232.

First actuator 240 is coupled to discharge end 248 of first conduit 232. Discharge end 248 of first conduit 232 is moved by activation and withdrawal of first actuator 240. First actuator 240 is configured to adjust discharge end 248 of first conduit 232 between a position where discharge end 248 of first conduit 232 is atop upper surface 186 of compression table 42 and a position where discharge end 248 of first conduit 232 is above die 178 of compression table 42. First actuator 240 may be hydraulic-activated, magnetic-activated, or pneumatic-activated. Operation of first actuator 240 is controlled by controller 34. First vibration unit 236 is coupled to first actuator 240 and is configured to vibrate first dosing unit 22. First vibration unit 236 preferably includes a DC motor having a voltage between 6V and 24V. In some configurations, the DC motor has a voltage between 12V and 24V. First vibration unit 236 may be capable of speeds between 0 and 10,000 RPM. In some configurations, first vibration unit 236 may be capable of speeds between 3,000 and 10,000 RPM. First vibration unit 236 may include an off-centered load between 0.5 grams and 25 grams on its shaft. Operation of first vibration unit 236 is controlled by controller 34. Vibration caused by first vibration unit 236 may be adjustable and may correspond to contents of the first powdered ingredient. In some configurations, the vibration unit may be replaced by a rotary force feeder.

Second dosing unit 26, shown in FIGS. 1-4, 6, and 7, includes a second conduit 256, a second vibration unit 260, and a second actuator 264. Second dosing unit 26 is configured to be placed atop upper surface 186 of compression table 42. Second dosing unit 26 guides a second powdered ingredient (not shown) which may be a second powdered or granulated inactive pharmaceutical ingredient, a second powdered or granulated active pharmaceutical ingredient, a second powdered or granulated blend of active pharmaceutical ingredients, or a second powdered or granulated blend of active pharmaceutical ingredients and inactive ingredients. The second powdered ingredient is dispensed into an intake end 268 of second conduit 256. The second powdered ingredient may be dispensed by an automated hopper or a manually-controlled hopper, or may be manually dispensed by the user. Intake end 268 of second conduit 256 may be configured as a funnel.

In some embodiments, second dosing unit 26 may include second pre-pressing sensor 416 configured to detect composition data of second powdered ingredient as first powdered ingredient flows through first dosing unit 22. Controller 34 receives the detected composition data from second pre-pressing sensor 416. Controller 34 then determines at least one of potency, water content, impurities, or a presence of polymorphs of second powdered ingredient from data collected by second pre-pressing sensor 416. Second pre-pressing sensor 416 may be any satisfactory analytical technology, such as an ultraviolet-visible spectrometer, a near-infrared spectrometer, a Raman spectrometer, a fluorescence spectrometer, a refractometer, or a micro-indentation sensor. Second pre-pressing sensor 416 is connected to and operably sends sensing signals to controller 34. Second pre-pressing sensor 416 provides in-line identification and composition of raw material powders, which may be used to adjust the tablet press process to produce tablets/layers of accurate dose and composition, based on the data detected by second pre-pressing sensor 416.

Second conduit 256 is configured to allow the second powdered ingredient to flow through second conduit 256 from intake end 268 to a discharge end 272. Discharge end 272 of second conduit 256 includes an opening 276 (shown in FIG. 7) that is configured to allow the second powdered ingredient to flow into die 178 of compression table 42 when discharge end 272 is placed above die 178. In addition, discharge end 272 of second conduit 256 is configured such that the second powdered ingredient is unable to pass between discharge end 272 of second conduit 256 and upper surface 186 of compression table 42 when discharge end 272 of second conduit 256 is in contact with upper surface 186 of compression table 42, thus preventing the second powdered ingredient from flowing out of second conduit 256.

Second actuator 264 is coupled to discharge end 272 of second conduit 256. Discharge end 272 of second conduit 256 is moved by activation and withdrawal of first actuator 64. Second actuator 264 is configured to adjust discharge end 272 of second conduit 256 between a position where discharge end 272 of second conduit 256 is atop compression table 42 and a position where discharge end 272 of second conduit 256 is above die 178 of compression table 42. Second actuator 264 may be hydraulic-activated, magnetic-activated, or pneumatic-activated. Operation of second actuator 264 is controlled by controller 34. Second vibration unit 260 is coupled to second actuator 264 and is configured to vibrate second dosing unit 26. Second vibration unit 260 may be a DC motor having a voltage between 6V and 24V. In some configurations, the DC motor has a voltage between 12V and 24V. Second vibration unit 260 may be capable of speeds between 0 and 10,000 RPM. In some configurations, second vibration unit 260 may be capable of speeds between 3,000 and 10,000 RPM. Second vibration unit 260 may include an off-centered load between 0.5 grams and 25 grams on its shaft. Operation of second vibration unit 260 is controlled by controller 34. Vibration caused by second vibration unit 260 may be adjustable and may correspond to contents of the second powdered ingredient. In some configurations, the vibration unit may be replaced by a rotary force feeder.

Although the embodiment shown in FIGS. 1-4, 6, and 7 includes first and second dosing units 22, 26, it should be understood that pharmaceutical apparatus 10 may include additional dosing units. The additional dosing units may be configured to hold additional powdered ingredients, which may include active pharmaceutical ingredients, inactive ingredients, or a mixture thereof.

A&QC unit 30 includes a plurality of stations 280, including a receiving station 284, an alignment station 288, at least one intermediate station 292, and a discharge station 296, and a transfer mechanism 300, as shown in FIGS. 1-4. A&QC unit 30 is physically separated from tablet press 18 to ensure that features of tablet press 18 and first and second dosing units 22, 26 (e.g., first and second vibration units 236, 260) do not affect the operation of plurality of stations 280. Each station of plurality of stations 280 is fixed at a location on A&QC unit 30. A&QC unit 30 also includes a tablet holding slot 304 coupled to transfer mechanism 300. Transfer mechanism 300 is configured to transfer tablet holding slot 304 to each station of plurality of stations 280. Tablet holding slot 304 is configured to move independently of plurality of stations 280. A&QC unit 30 may include a plurality of tablet holding slots, such that multiple pharmaceutical tablets may traverse A&QC unit 30 concurrently. Each tablet holding slot in the plurality of tablet holding slots may be evenly spaced around A&QC unit 30 (e.g., when A&QC unit 30 is in a round configuration, as in FIGS. 1-4) or along A&QC unit 30 (e.g., when A&QC unit 30 is in a rectangular configuration).

Tablet holding slot 304 includes a tablet actuator 308 therein. Tablet actuator 308 is operated by controller 34. Tablet holding slot 304 is configured to hold pharmaceutical tablet 14 in an execution position 312, such that each execution device 316 of each intermediate station of at least one intermediate station 292 may properly access pharmaceutical tablet 14 when pharmaceutical tablet 14 is in execution position 312. Execution position 312 is adjacent to tablet actuator 308 within tablet holding slot 304. Pharmaceutical tablet 14 is placed in execution position 312 within tablet holding slot 304 by forces acting on pharmaceutical tablet 14 as transfer mechanism 300 transfers tablet holding slot 304.

Receiving station 284 is located adjacent to second end 228 of discharge chute 54 of tablet press 18. Receiving station 284 is configured such that pharmaceutical tablet 14 traversing discharge chute 54 continues into tablet holding slot 304 when tablet holding slot 304 is present at receiving station 284.

Alignment station 288 is located adjacent to receiving station 284. The movement of tablet holding slot 304 from receiving station 284 to alignment station 288 places pharmaceutical tablet 14 in execution position 312.

A first intermediate station 332 of at least one intermediate station 292 is adjacent to alignment station 288. Further intermediate stations may be located adjacent to a preceding intermediate station of at least one intermediate station 292 (e.g., a second intermediate station 344 is adjacent to first intermediate station 332 and a third intermediate station 352 is adjacent to second intermediate station 344).

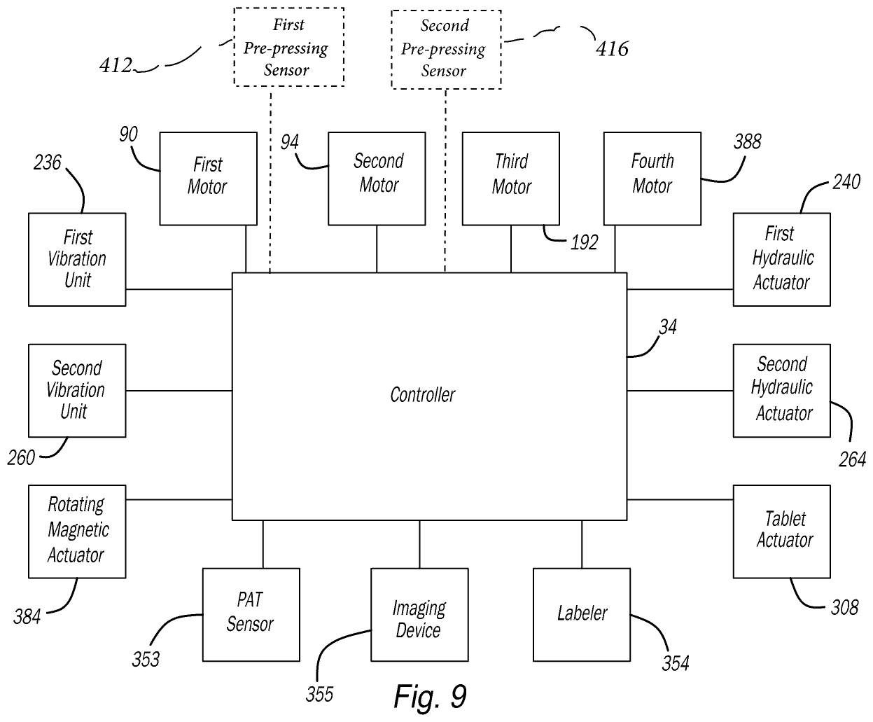

In some configurations, the number of intermediate stations 292 may be higher than those shown in FIGS. 1-4. In some configurations, the number of intermediate stations 292 may be lower than those shown in FIGS. 1-4. The order in which intermediate stations 292 present execution devices 316 may vary in some configurations. Execution device 316 at each intermediate station of at least one intermediate station 292 may be configured to inspect pharmaceutical tablet 14 or may be configured to provide a tablet identifier to pharmaceutical tablet 14. Execution device 316 at each intermediate station of at least one intermediate station 292 may be any one of a PAT sensor 353, a labeler 354, an imaging device 355, a weighing device 357 (as shown in FIG. 8), a hardness tester (not shown), or another relevant device.

In some configurations, an intermediate station may include PAT sensor 353 as execution device 316, such as first intermediate station 332 in FIGS. 1-4. PAT sensor 353 is configured to detect composition data of pharmaceutical tablet 14 when pharmaceutical tablet 14 is present at an intermediate station including PAT sensor 353 as execution device 316 (e.g., first intermediate station 332 in FIGS. 1-4). Controller 34 receives the detected composition data from PAT sensor 353. Controller 34 then determines at least one of potency, hardness, water content, impurities, or a presence of polymorphs of pharmaceutical tablet 14 from data collected by PAT sensor 353. PAT sensor 353 may be any satisfactory analytical technology, such as an ultraviolet-visible spectrometer, a near-infrared spectrometer, a Raman spectrometer, a fluorescence spectrometer, a refractometer, or a micro-indentation sensor. PAT sensor 353 is connected to and operably sends sensing signals to controller 34.

In some configurations, an intermediate station may include labeler 354 as execution device 316, such as second intermediate station 344 in FIGS. 1-4. Labeler 354 is configured to mark pharmaceutical tablet 14 when pharmaceutical tablet 14 is present at an intermediate station including labeler 354 as execution device 316 (e.g., third intermediate station 352 in FIGS. 1-4). Labeler 354 is configured to mark pharmaceutical tablet 14 with the tablet identifier. The tablet identifier may comprise any of text, numbers, or symbols, which may assist the user or a patient in identifying pharmaceutical tablet 14. The tablet identifier may be provided by the user to controller 34 or from a stored database within controller 34. The tablet identifier may include an active ingredient dosage of pharmaceutical tablet 14, an active ingredient of pharmaceutical tablet 14 (such as a commercial or a chemical name of the active ingredient), a barcode, a QR code, or another beneficial identifier. Labeler 354 is preferably an inkjet printer, by way of nonlimiting example. The inkjet printer may be capable of print speeds up to 70 m/min (230 fpm). The print quality from the inkjet printer may have a resolution of 600×300 dots per inch. The inkjet printer may be capable of printing up to 8 lines of programmable text on a pharmaceutical tablet 14. The inkjet printer may be capable of printing from up to 7 inches from a surface of pharmaceutical tablet 14. In some configurations, labeler 354 may etch writing into pharmaceutical tablet 14. Labeler 354 receives direction from and is controlled by controller 34.

In some configurations, an intermediate station may include imaging device 355 as execution device 316, such as third intermediate station 352 in FIGS. 1-4. Imaging device 355 is configured to capture an image of pharmaceutical tablet 14 when pharmaceutical tablet 14 is present at an intermediate station including imaging device 355 as execution device 316 (e.g., second intermediate station 344 in FIGS. 1-4). Imaging device 355 may be configured such that the image captured by imaging device 355 may allow controller 34 to determine visual characteristics (e.g., color, tablet identifiers, defects, non-uniformities), dimensions of pharmaceutical tablet 14 or of any layer of pharmaceutical tablet 14, including but not limited to a height 356, diameter, radius, and curvature of pharmaceutical tablet 14, a thickness 359 of a first powdered ingredient layer 362 of pharmaceutical tablet 14, and a thickness 365 of a second powdered ingredient layer 368 of pharmaceutical tablet 14, and hardness of pharmaceutical tablet 14 using image processing techniques. In some configurations, the image captured by imaging device 355 may allow controller 34 to determine dimensions of any additional layers of a pharmaceutical tablet using image processing techniques. Imaging device 355 may provide images as either 2D and/or 3D. Imaging device 355 may be a camera. In some example embodiments, imaging device 355 may be a 3D scanner. As an example, imaging device 355 may include a complementary metal-oxide-semiconductor sensor (CMOS), which may have an optical format of 1/1.9″. A resolution of a camera as imagining device 355 may be a minimum of five million active pixels, such as 2592×1944 for example. Each pixel size may be 2.4 μm×2.4 μm. The active sensor area of imaging device 355 may be 6.22 mm×4.67 mm. The camera shutter type of imaging device 355 may be electronic rolling. Imaging device 355 sensitivity may be 425 mV@1/30 s−f/5.6. Imaging device 355 may have a spectral response of 380-650 nm with an infrared-cut filter. In embodiments in which the imaging device 355 is a 3D scanner, the imaging device 355 may be configured to scan the entirety of a pharmaceutical tablet, such that a plurality of dimensions (e.g., thickness, thickness of layers, diameter) may be detected and/or calculated from the resulting scan. Imaging device 355 is connected to and operably sends sensed digital image signals to controller 34.

Some configurations may include weighing device 357 as an execution device 316. The weighing device 357 may be a scale. Weighing device 357 is configured to weigh pharmaceutical tablet 14 when pharmaceutical tablet 14 is present at an intermediate station including weighing device 357 as execution device 316. Weighing device 357 is connected to and operably sends sensed weight signals to controller 34.

Some configurations may include a hardness tester as an execution device 316. The hardness tester is configured to contact pharmaceutical tablet 14 and to gather hardness data of pharmaceutical tablet 14 when the pharmaceutical tablet is present at an intermediate station including the hardness tester as execution device 316. The hardness tester is connected to and operably sends sensed hardness signals to controller 34.

Discharge station 296 is adjacent to a final intermediate station (e.g., third intermediate station 352 in FIGS. 1-4). At discharge station 296, tablet actuator 308 ejects pharmaceutical tablet 14 to one of an accepted tablet container 372 and a waste tablet container 376. Between tablet actuator 308 and accepted and waste tablet containers 372, 376, discharge station 296 includes a discharge gate 380 coupled to a rotating magnetic actuator 384. Rotating magnetic actuator 384 is operated by controller 34. Discharge gate 380 is configured to allow pharmaceutical tablet 14 to be placed in one of accepted tablet container 372 and waste tablet container 376. Discharge gate 380 accomplishes this by being positioned such that discharge gate 380 blocks pharmaceutical tablet 14 from entering an incorrect container of accepted tablet container 372 and waste tablet container 376, as determined by controller 34. In some configurations, discharge station 296 may be configured to also allow pharmaceutical tablet 14 to instead be ejected to a retaining container (not shown).

Transfer mechanism 300 is configured to transfer tablet holding slot 304 by a fourth motor 388 coupled to transfer mechanism 300. Operation of fourth motor 388 is controlled by controller 34. In configurations featuring a round A&QC unit 30, such as the embodiment shown in FIGS. 1-4, transfer mechanism 300 comprises a rotary disk 392. Rotary disk 392 is rotated by fourth motor 388 coupled to rotary disk 392. In some configurations featuring a linear A&QC unit 30, transfer mechanism 300 may comprise a conveyor line configured to traverse a length of A&QC unit 30.

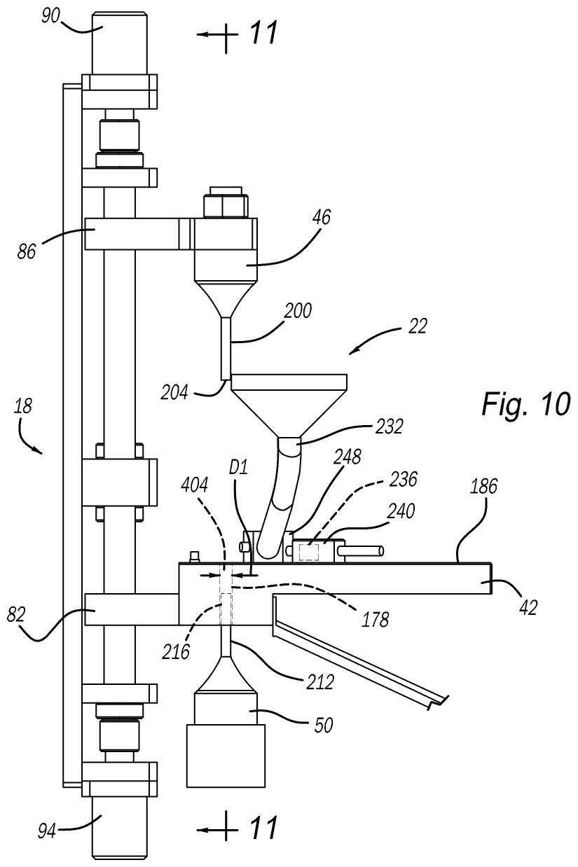

Pharmaceutical apparatus 10 also includes programmable controller 34. As shown in FIG. 9, controller 34 of the embodiment shown in FIGS. 1-4 is in communication with first, second, third, and fourth motors 90, 94, 192, 388, first and second vibration units 236, 260, first and second actuators 240, 264, tablet actuator 308, PAT sensor 353, labeler 354, imaging device 355, and rotating magnetic actuator 384. Controller 34 may be in communication with first pre-pressing sensor 412 and second pre-pressing sensor 416. In configurations including weighing device 357, the controller 34 will also be in communication with weighing device 357.

Controller 34 includes a microprocessor for operably running the programmable software instruction, non-transient ROM or RAM memory, an input component such as a keyboard or switches, an output such as a display screen, a power supply, an electrical circuit connecting the electronic hardware, and other suitable hardware components. Controller 34 may include one or more interface and communications circuits, including wired or wireless interfaces that are connected to a local area network (LAN), the Internet, a wide area network (WAN), a controller area network (CAN), or combinations thereof. The electrical circuit of controller 34 is connected to receive signals from any of PAT sensor 353, labeler 354, imaging device 355, and weighing device 357, and to energize/deenergize the actuators. The electrical circuit of controller 34 may be connected to receive signals from first pre-pressing sensor 412 and second pre-pressing sensor 416. The methods described in this application regarding controller 34 may be partially or fully implemented by a special purpose computer created by configuring a general purpose computer to execute one or more particular functions embodied in computer programs.

Controller 34 may include a user interface (e.g., a graphical user interface displayed on a monitor, a laptop, or a smart phone etc.) which may be used by a user to provide information to the controller 34. The interface may include user input fields to allow user to provide specific information in a correct format, such as proper units. If an issue with a particular user input is detected, the interface may display warning messages to inform the user.

Controller 34 is configured to receive details of a pharmaceutical tablet order to be manufactured from the user. The pharmaceutical tablet order is customized by the user to suit needs of a specific patient based on the details of the pharmaceutical tablet order. The details of the pharmaceutical tablet order may include any of an active pharmaceutical ingredient concentration in the first powdered ingredient, an active pharmaceutical ingredient concentration in the second powdered ingredient, a density of the first powdered ingredient, a density of the second powdered ingredient, a particle size of the first powdered ingredient, a particle size of the second powdered ingredient, a color of the first powdered ingredient, a color of the second powdered ingredient, the tablet identifier, a desired dosage of each active pharmaceutical ingredient of pharmaceutical tablet 14, height 356 of pharmaceutical tablet 14, a size of pharmaceutical tablet 14, thickness 359 of first powdered ingredient layer 362 of pharmaceutical tablet 14, thickness 365 of second powdered ingredient layer 368 of pharmaceutical tablet 14, a weight of pharmaceutical tablet 14, a hardness of pharmaceutical tablet 14 and a number of pharmaceutical tablets 14 to be produced. Details of the pharmaceutical tablet order which pertain to information of first and second powdered ingredients (e.g., active pharmaceutical ingredient concentration in the first powdered ingredient, active pharmaceutical ingredient concentration in the second powdered ingredient, density of the first powdered ingredient, density of the second powdered ingredient, particle size of the first powdered ingredient, particle size of the second powdered ingredient) may alter based on detected values of said details by the first and second pre-pressing sensors 412, 416. The active adjustments of said details may allow pharmaceutical apparatus 10 to better control the dosage of pharmaceutical tablet 14 of each active ingredient. The user may also provide an allowable error range or tolerance for each of the details of the pharmaceutical tablet order.

Because the details of the pharmaceutical tablet order may be defined by the user, the patient receiving the tablets may be able to receive care better suited for their specific situation. For example, a subsequent patient may require a different pharmaceutical tablet from a current patient (i.e., different dosage of an active ingredient(s), an overall tablet size, drug release profiles, drug product, or active pharmaceutical ingredients of the pharmaceutical tablets). By altering the details of the subsequent patient's pharmaceutical tablet order, the user can customize the pharmaceutical tablets for the subsequent patient while not affecting the current patient's pharmaceutical tablets. Pharmaceutical apparatus 10 allows a seamless transition from manufacturing the current patient's pharmaceutical tablets to manufacturing the subsequent patient's pharmaceutical tablets.

Pharmaceutical tablet 14 produced by pharmaceutical apparatus 10 includes first powdered ingredient layer 362 and second powdered ingredient layer 368, as shown in FIG. 5. First powdered ingredient layer 362 and second powdered ingredient layer 368 are pressed together to form pharmaceutical tablet 14. In some configurations, a pharmaceutical tablet produced may include additional layers pressed together to form the pharmaceutical tablet.

The customization available within the pharmaceutical tablet order allows for multiple pharmaceutical tablet orders to maintain a common pharmaceutical tablet size and/or dimensions, even if the desired dosage of each active pharmaceutical ingredient varies between pharmaceutical tablet orders. In other words, a dosage of an active pharmaceutical ingredient ordered in a first pharmaceutical tablet order may differ from a dosage of the active pharmaceutical ingredient in a second pharmaceutical tablet order, but the size of the pharmaceutical tablets of the first pharmaceutical tablet order and the second pharmaceutical tablet order will be equivalent. Thickness 359 and/or weight of first powdered ingredient layer 362 and thickness 365 and/or weight of second powdered ingredient layer 368 may vary between the first pharmaceutical tablet order and the second pharmaceutical tablet order to allow the difference to be identifiable by the patient in such a situation. Additionally, successive pharmaceutical tablet orders may include varying active pharmaceutical ingredients and/or a different number of layers of the pharmaceutical tablets.

An additional possibility includes repeating a powdered ingredient in multiple layers of a multi-layer pharmaceutical tablet. For example, a three-layer tablet may utilize an inactive powered ingredient blend on a top and bottom layer with a middle layer including an active pharmaceutical ingredient blend. A further example may include another exemplary multi-layer pharmaceutical tablet including a first active pharmaceutical ingredient in a first layer of the exemplary pharmaceutical tablet and a second active pharmaceutical ingredient in a second layer of the exemplary pharmaceutical tablet, such that a patient may ingest multiple prescriptions in a single pharmaceutical tablet. In yet another example, pharmaceutical apparatus 10 may be capable of producing a single-layer pharmaceutical tablet by utilizing a single powdered ingredient.

With reference to FIGS. 1-4 and 10-13, operation of pharmaceutical apparatus 10 will now be described in detail. To begin manufacturing of pharmaceutical tablet 14, the user first inputs the details of the pharmaceutical tablet order into controller 34. Using the details of the pharmaceutical tablet order, controller 34 calculates a volume of the first powdered ingredient and a volume of the second powdered ingredient to be included in pharmaceutical tablet 14. The volume of the first powdered ingredient and the volume of the second powdered ingredient to be included in the pharmaceutical tablet 14 may be adjusted based on characteristics of the first and second powdered ingredients detected by the first and second pre-pressing sensors 412, 416.

Using the calculated volume of the first powdered ingredient, the volume of the second powdered ingredient, and diameter D1 of die 178 of compression table 42, controller 34 determines a fill depth of the first powdered ingredient and a fill depth of the second powdered ingredient.

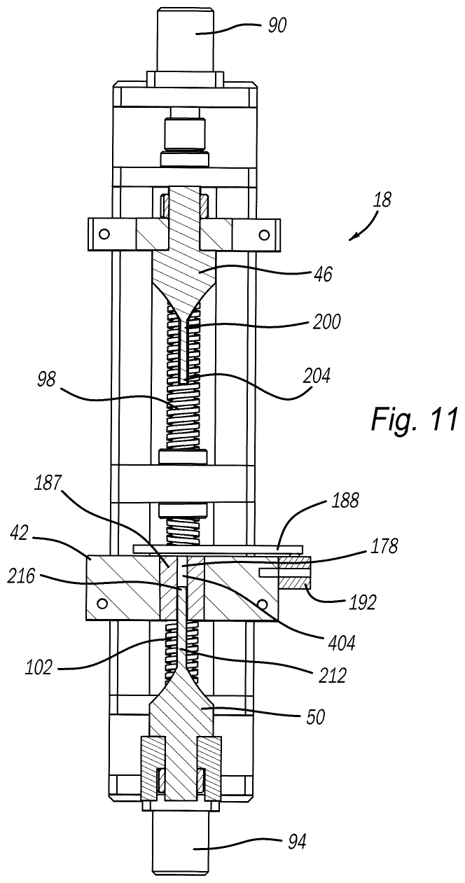

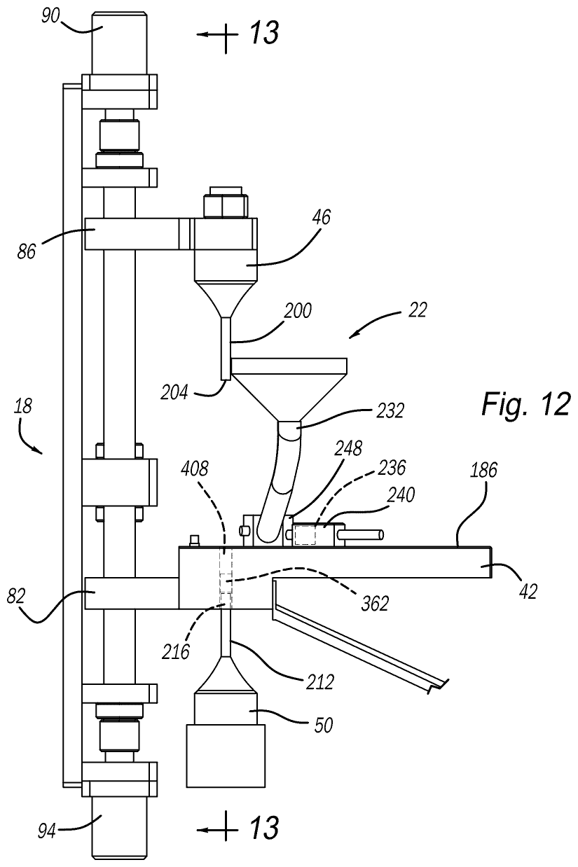

Second motor 94 rotates second ball screw 102 to place compression table mount-plate 82 in a position in which first end 216 of lower compressing rod 212 is received in die 178 of compression table 42, such as in FIGS. 10 and 11. Compression table 42 is placed in a position such that a height of a first fill space 404, which is a space within die 178 of compression table 42 above lower compressing rod 212, is equal to the fill depth of the first powdered ingredient.

First actuator 240 activates and drives discharge end 248 of first conduit 232 to the position above die 178 of compression table 42. When discharge end 248 of first conduit 232 is positioned above die 178 of compression table 42, the first powdered ingredient begins to flow into die 178 through discharge end 248 of first conduit 232. With discharge end 248 of first conduit 232 being positioned above die 178 of compression table 42, first vibration unit 236 vibrates first dosing unit 22 resulting in improved flow of the first powdered ingredient through first dosing unit 22 into die 178 of compression table 42. After the first powdered ingredient has filled first fill space 404, first actuator 240 retracts and moves discharge end 248 of first conduit 232 atop upper surface 186 of compression table 42 to stop the flow of the first powdered ingredient.

First motor 90 then rotates first ball screw 98 such that first end 204 of upper compressing rod 200 penetrates die 178 of compression table 42. First end 204 of upper compressing rod 200 penetrates die 178 of compression table 42 to a first depth, as determined by controller 34, to compress the first powdered ingredient present in first fill space 404 into first powdered ingredient layer 362 of pharmaceutical tablet 14. The first depth is calculated such that first powdered ingredient layer 362 will be compacted to a desired density corresponding to the details of the pharmaceutical tablet order.

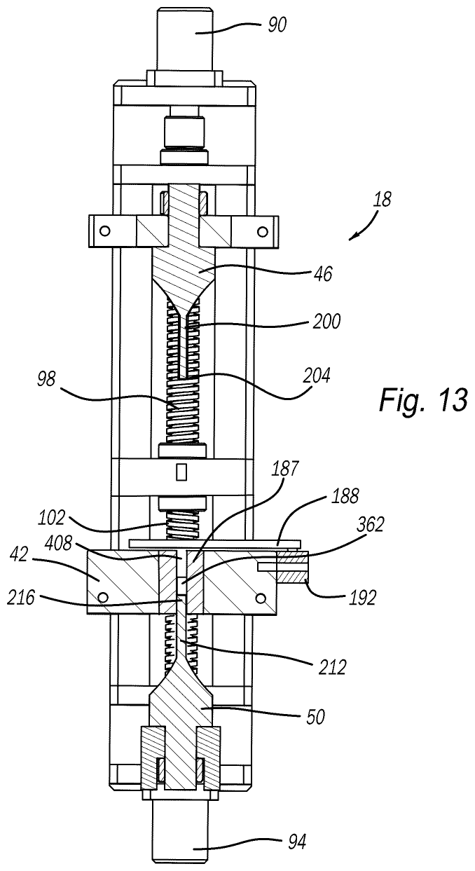

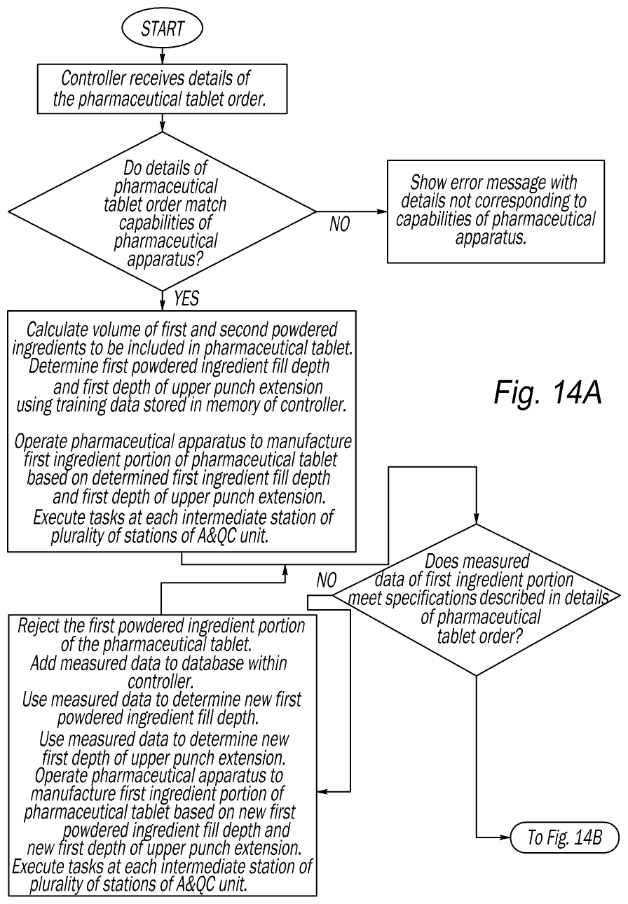

While first end 204 of upper compressing rod 200 remains within die 178 of compression table 42, second motor 94 rotates second ball screw 102 and raises compression table mount-plate 82 such that a height of a second fill space 408, which is a space within die 178 of compression table 42 above the first powdered ingredient layer 362, is equal to the fill depth of the second powdered ingredient. First motor 90 then rotates first ball screw 98 and raises upper punch 46, thus removing upper compressing rod 200 from second fill space 408. This position is shown in FIGS. 12 and 13.

Second actuator 264 activates and drives discharge end 272 of second conduit 256 to a position above die 178 of compression table 42. When discharge end 272 of second conduit 256 is positioned above die 178 of compression table 42, the second powdered ingredient begins to flow into die 178 of compression table 42 through discharge end 272 of second conduit 256. With discharge end 272 of second conduit 256 being positioned above die 178 of compression table 42, second vibration unit 260 vibrates second dosing unit 26 resulting in improved flow of the second powdered ingredient through second dosing unit 26 into die 178 of compression table 42. After the second powdered ingredient has filled second fill space 408, second actuator 264 retracts and moves discharge end 272 of second conduit 256 atop upper surface 186 of compression table 42 to stop the flow of the second powdered ingredient.

First motor 90 rotates first ball screw 98 such that first end 204 of upper compressing rod 200 penetrates die 178 of compression table 42. First end 204 of upper compressing rod 200 penetrates die 178 of compression table 42 to a second depth, as determined by controller 34, to compress the second powdered ingredient present in second fill space 408 into second powdered ingredient layer 368 of pharmaceutical tablet 14. The compression of the second powdered ingredient causes second powdered ingredient layer 368 and first powdered ingredient layer 362 to form pharmaceutical tablet 14. The second depth is calculated such that second powdered ingredient layer 368 will be compacted to a desired density corresponding to the details of the pharmaceutical tablet order.

First motor 90 rotates first ball screw 98 and raises upper punch 46, thus removing upper compressing rod 200 from die 178 of compression table 42. Second motor 94 then rotates second ball screw 102 and lowers compression table mount-plate 82 until pharmaceutical tablet 14 is ejected from die 178 of compression table 42. With pharmaceutical tablet 14 ejected from die 178 of compression table 42, third motor 192 rotates wiper 188 such that wiper 188 pushes pharmaceutical tablet 14 in the direction of first aperture 182 of compression table 42. When pharmaceutical tablet 14 is pushed to first aperture 182 of compression table 42, pharmaceutical tablet 14 falls through first aperture 182 of compression table 42 to first end 224 of discharge chute 54 below first aperture 182 of compression table 42.

Pharmaceutical tablet 14 traverses discharge chute 54 in a direction toward second end 228 of discharge chute 54 due to the downward slope of discharge chute 54. Pharmaceutical tablet 14 continues past second end 228 of discharge chute 54 and is delivered to tablet holding slot 304 when tablet holding slot 304 is present at receiving station 284 of A&QC unit 30.

After pharmaceutical tablet 14 is received in tablet holding slot 304, fourth motor 388 activates transfer mechanism 300 to transfer tablet holding slot 304 holding pharmaceutical tablet 14 to alignment station 288. In the embodiment shown in FIGS. 1-4, fourth motor 388 first rotates transfer mechanism 300 36 degrees. As transfer mechanism 300 advances tablet holding slot 304 to alignment station 288, rotational forces caused by movement of transfer mechanism 300 moves pharmaceutical tablet 14 to execution position 312 by sliding within tablet holding slot 304.

Fourth motor 388 activates transfer mechanism 300 to transfer tablet holding slot 304 holding pharmaceutical tablet 14 to first intermediate station 332. In the embodiment shown in FIGS. 1-4, fourth motor 388 rotates transfer mechanism 300 36 degrees to place tablet holding slot 304 holding pharmaceutical tablet 14 at first intermediate station 332.

In the embodiment shown in FIGS. 1-4, first intermediate station 332 of at least one intermediate station 292 includes PAT sensor 353 as execution device 316. PAT sensor 353 gathers the composition data of pharmaceutical tablet 14. The composition data is provided to controller 34 and controller 34 determines at least one of potency, hardness, water content, impurities, or the presence of polymorphs of pharmaceutical tablet 14 through use of the composition data. The chemical composition data of pharmaceutical tablet 14 is stored in memory of controller 34.

Fourth motor 388 activates transfer mechanism 300 to transfer tablet holding slot 304 holding pharmaceutical tablet 14 to second intermediate station 344. In the embodiment shown in FIGS. 1-4, fourth motor 388 rotates transfer mechanism 300 36 degrees to place tablet holding slot 304 holding pharmaceutical tablet 14 at second intermediate station 344.

In the embodiment shown in FIGS. 1-4, second intermediate station 344 of at least one intermediate station 292 includes labeler 354 as execution device 316. Labeler 354 marks pharmaceutical tablet 14 with the tablet identifier.

Fourth motor 388 activates transfer mechanism 300 to transfer tablet holding slot 304 holding pharmaceutical tablet 14 to third intermediate station 352. In the embodiment shown in FIGS. 1-4, fourth motor 388 rotates transfer mechanism 300 36 degrees to place tablet holding slot 324 holding pharmaceutical tablet 14 at third intermediate station 352.

In the embodiment shown in FIGS. 1-4, third intermediate station 352 of at least one intermediate station 292 includes imaging device 355 as execution device 316. Imaging device 355 obtains an image of pharmaceutical tablet 14. The image is provided to controller 34 and controller 34 determines visual characteristics (e.g., color, tablet identifiers, defects, non-uniformities), dimensions of pharmaceutical tablet 14 or of any layer of pharmaceutical tablet 14, including but not limited to a height 356, diameter, radius, and curvature of pharmaceutical tablet 14, a thickness 359 of a first powdered ingredient layer 362 of pharmaceutical tablet 14, and a thickness 365 of a second powdered ingredient layer 368 of pharmaceutical tablet 14, and hardness of pharmaceutical tablet 14 through image processing techniques. The image captured by imaging device 355 and the processed data are stored in memory of controller 34.

In configurations including weighing device 357 as the execution device of an intermediate station, weighing device 357 gathers weight information of the pharmaceutical tablet and provides the weight information to controller 34. The weight information may be used in combination with size data processed by controller 34 to determine density of each layer of pharmaceutical tablet 14. The weight information would then be stored in the memory of controller 34.

In configurations including the hardness tester as the execution device of an intermediate station, the hardness tester gathers hardness data of the pharmaceutical tablet and provides the hardness data to controller 34. The hardness data would then be stored in memory of controller 34.

Controller 34 determines whether characteristics of pharmaceutical tablet 14 (e.g., potency of pharmaceutical tablet 14, thickness 359 of first powdered ingredient layer 362 of pharmaceutical tablet 14, thickness 365 of second powdered ingredient layer 368 of pharmaceutical tablet 14, density of first powdered ingredient layer 362 of pharmaceutical tablet 14, density of second powdered ingredient later 368 of pharmaceutical tablet 14, height 356 of pharmaceutical tablet 14, the weight of pharmaceutical tablet 14, and/or hardness of the pharmaceutical tablet 14) are within the allowable error range or tolerance of the details of the pharmaceutical tablet order. If characteristics of the pharmaceutical tablet 14 are within the allowable error range of the details of the pharmaceutical tablet order, then pharmaceutical tablet 14 is accepted. If characteristics of pharmaceutical tablet 14 are not within the allowable error range of the details of the pharmaceutical tablet order, then pharmaceutical tablet 14 is rejected. The details of the pharmaceutical tablet order to be considered when making such a determination may be provided by the user.

Fourth motor 388 activates transfer mechanism 300 to transfer tablet holding slot 304 holding pharmaceutical tablet 14 to discharge station 296. If pharmaceutical tablet 14 is accepted, then controller 34 directs rotating magnetic actuator 384 to place discharge gate 380 between pharmaceutical tablet 14 and waste tablet container 376. In such a case, pharmaceutical tablet 14 may only be ejected to accepted tablet container 372. If pharmaceutical tablet 14 is rejected, then controller 34 directs rotating magnetic actuator 384 to place discharge gate 380 between completed pharmaceutical tablet 14 and accepted tablet container 372. In such a case, pharmaceutical tablet 14 may only be ejected to waste tablet container 376. When discharge gate 380 is in the proper position as determined by controller 34, tablet actuator 308 ejects pharmaceutical tablet 14 into the designated container of accepted tablet container 372 and waste tablet container 376. In configurations including the retaining container, the pharmaceutical tablet 14 may be ejected to the retaining container, in situations such as when the pharmaceutical tablet 14 is to be held for further post-manufacturing inspection and when the pharmaceutical tablet 14 was manufactured as part of a calibration process.

During operation of pharmaceutical apparatus 10, the microprocessor of controller 34 will operate an automated sensor feedback loop that can adjust operation parameters for a subsequent pharmaceutical tablet within the same pharmaceutical tablet order. These operation parameters include the first depth and the second depth that upper punch 46 extends into die 178, and the fill depths of the first powdered ingredient and the second powdered ingredient. The operation parameters may also include vibration characteristics, pharmaceutical apparatus 10 timing characteristics, and a speed of first motor 90. Throughout the feedback loop, controller 34 determines whether characteristics of pharmaceutical tablet 14 are within the allowable error range or tolerance of the details of the pharmaceutical tablet order. The details of the pharmaceutical tablet order to be considered when making such a determination may be provided by the user. The allowable error range or tolerance of the details of the pharmaceutical tablet order may be further divided into an acceptable range and an adjust range. The acceptable range is a tighter range to a target value for each of the details of the pharmaceutical tablet order. The adjust range includes values not within the acceptable range for each of the details of the pharmaceutical tablet order, but still within the allowable error range or tolerance of the details of the pharmaceutical tablet order.

If characteristics of pharmaceutical tablet 14 are within the acceptable range, then pharmaceutical tablet 14 is accepted and no changes are made to the operation parameters of pharmaceutical apparatus 10.

If characteristics of pharmaceutical tablet 14 are within the adjust range, then pharmaceutical tablet 14 is accepted, but an operation parameter of pharmaceutical apparatus 10 is adjusted such that a subsequent pharmaceutical tablet is manufactured within the acceptable range. For example, when the pharmaceutical tablet 14 is found to have a density within the adjust range, but lower than the values of the acceptable range, then the first depth and/or the second depth may be extended further into the die 178 during manufacture of the subsequent pharmaceutical tablet to create a denser (i.e., more compact) layer(s) of the subsequent pharmaceutical tablet when compared to the current pharmaceutical tablet.

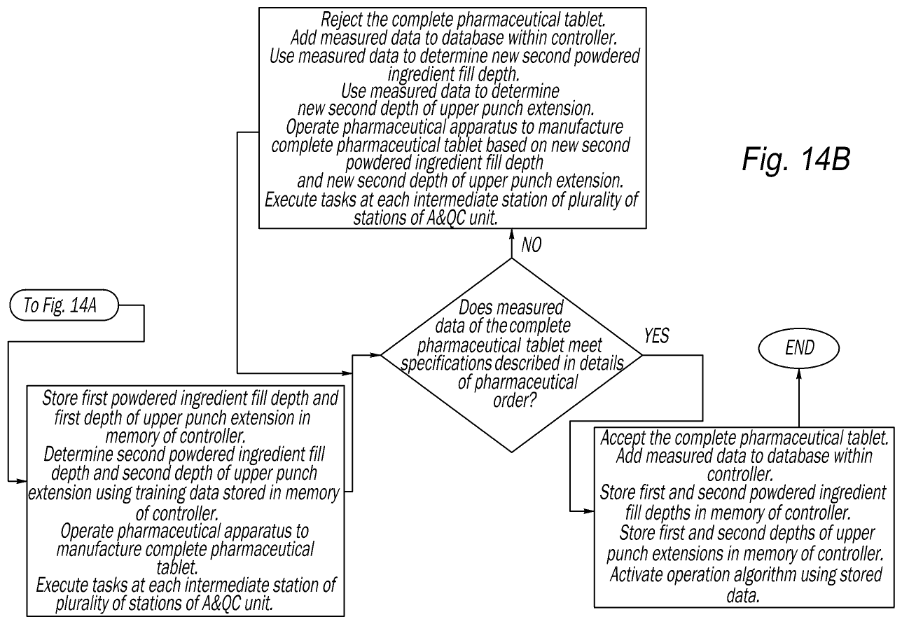

If characteristics of pharmaceutical tablet 14 are not within the allowable error range of the details of the pharmaceutical tablet order, then pharmaceutical tablet 14 is rejected. Pharmaceutical apparatus 10 will then be recalibrated in accordance with the calibration method described in FIGS. 14A and 14B.

Computer software instructions for calibrating pharmaceutical apparatus 10 are disclosed in FIGS. 14A and 14B. These computer software instructions are stored in the in the non-transient memory and automatically run by the microprocessor of controller 34. The logic diagram and method steps of FIGS. 14A and 14B show a calibration method of pharmaceutical apparatus 10, including first manufacturing first powdered ingredient layer 362 of pharmaceutical tablet 14 and inspecting first powdered ingredient layer 362 of pharmaceutical tablet 14 throughout plurality of stations 280 of A&QC unit 30. If first powdered ingredient layer 362 of pharmaceutical tablet 14 is not within the allowable error range or tolerance of details of the pharmaceutical tablet order which relate to first powdered ingredient layer 362 of pharmaceutical tablet 14 (e.g., thickness 359 of first powdered ingredient layer 362), controller 34 adjusts the fill depth of the first powdered ingredient and/or the first depth which upper punch 46 extends into die 178 and the manufacturing and inspection processes of first powdered ingredient layer 362 of pharmaceutical tablet 14 are repeated. If first powdered ingredient layer 362 of pharmaceutical tablet 14 is within the allowable error range or tolerance of the details of the pharmaceutical tablet order which relate to first powdered ingredient layer 362 of pharmaceutical tablet 14, a complete pharmaceutical tablet 14 is manufactured and is inspected throughout plurality of stations 280 of A&QC unit 30.

If complete pharmaceutical tablet 14 is not within the allowable error range of the details of the pharmaceutical tablet order, controller 34 adjusts the fill depth of the second powdered ingredient and/or the second depth which upper punch 46 extends into die 178 and the manufacturing and inspection processes of pharmaceutical tablet 14 are repeated. If pharmaceutical tablet 14 is within specifications described by the details of the pharmaceutical tablet order, then pharmaceutical tablet 14 is accepted and operation of pharmaceutical apparatus 10 continues with stored fill depths of the first and second powdered ingredients and first and second depths which upper punch 46 extends into die 178.

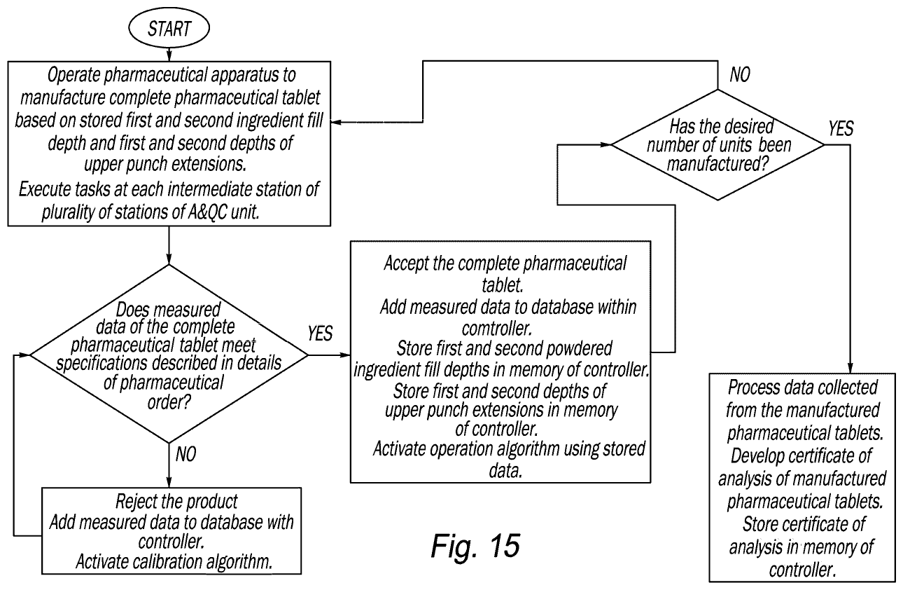

FIG. 15 discloses the computer software instructions for operation of pharmaceutical apparatus 10 throughout manufacture of a pharmaceutical tablet order. These computer software instructions are stored in the non-transient memory and run by the microprocessor of controller 34. The logic diagram and method steps of FIG. 15 direct pharmaceutical apparatus 10 to engage the calibration method described in FIGS. 14A and 14B when a pharmaceutical tablet 14 is rejected during operation of pharmaceutical apparatus 10. The logic diagram and method steps of FIG. 15 also direct pharmaceutical apparatus 10 to continually operate until all pharmaceutical tablets to be produced in the pharmaceutical tablet order have been produced. The operation of the automated sensor feedback loop previously described may be utilized within the method shown in FIG. 15 (i.e., pharmaceutical tablets produced within the adjust range are accepted but operation parameters of pharmaceutical apparatus 10 may be adjusted to reach the accepted range).

The United States Food and Drug Administration and similar regulatory bodies may require measurement of certain characteristics of medications, including drug product potency, uniformity, dissolution, impurities, water content, tablet hardness, visual defects/non-uniformity, and presence of positive drug product identifiers or markers, as traditionally performed using classical off-line methods of samples collected from the manufacturing process. In place of traditional off-line techniques, the above described apparatus and method utilizes in-line analytics to measure other tablet and/or individual layer properties, such as dimensional analysis by 2D or 3D imaging and/or laser scanning techniques (height, thickness, and width to determine volume or size), weight, density (as calculated from in-line dimensional analysis & weights), spectroscopic identification of raw materials (i.e., powdered ingredients entering the dosage unit), intermediate materials (i.e. layers) and the finished product (i.e. tablets), and 2D or 3D imaging and/or laser scanning techniques for assessment of visual appearance, uniformity, defects, and/or marker presence/accuracy. When used in combination, these various techniques allow for analytical identification of the materials present in various stages of manufacture (e.g., spectrometry of raw materials, layers, and finished tablets) as well as separate, visual identification of a tablet (e.g., visual confirmation of tablet identifier). These in-line techniques may then be correlated with those measured by traditional off-line methods to substantiate product specifications and release testing by in-line measurements to regulatory bodies.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. For example, pneumatic or hydraulic powered actuators, or other electromagnetic actuators, may be used instead of the present exemplary electric motor actuators, and vice versa, although some of the present benefits may not be fully realized. The pharmaceutical tablets described in the above disclosure may be of any suitable shape, including cylindrical with either a circular cross-sectional shape or an oval cross-sectional shape. Furthermore, additional or different software instruction steps can be employed, however, certain advantages may not be obtained. The features of one embodiment can be mixed and matched in any combination with other embodiments, and the claims may be multiply dependent on each other in any and all combinations. Such variations are not to be regarded as a departure from the disclosure, and all such modifications are intended to be included within the scope of the present disclosure.

What is claimed is:

1. A pharmaceutical apparatus comprising:
a tablet press configured to compress a first powdered ingredient and a second powdered ingredient into a multi-layer pharmaceutical tablet; and
an analytical and quality control table physically separated from the tablet press configured to receive the pharmaceutical tablet from the tablet press, the analytical and quality control table comprising at least one PAT sensor;
the PAT sensor comprises at least one of: an ultraviolet-visible spectrometer, a near-infrared spectrometer, a Raman spectrometer, a fluorescence spectrometer, a refractometer, or a micro-indentation sensor; and
wherein the at least one PAT sensor is configured to assist in determining a potency of a first layer with the first powdered ingredient and at least a second layer with the second powdered ingredient of the pharmaceutical tablet.

2. The pharmaceutical apparatus of claim 1, wherein the analytical and quality control table further comprises an imaging device configured to capture an image of each of the layers of the pharmaceutical tablet.

3. The pharmaceutical apparatus of claim 2, wherein the imaging device is configured such that the image of the pharmaceutical tablet is able to assist in determining hardness of each of the layers of the pharmaceutical tablet.

4. The pharmaceutical apparatus of claim 2, wherein the imaging device is configured such that the image of the pharmaceutical tablet is able to assist in determining at least one characteristic of each layer of the pharmaceutical tablet, the at least one characteristic selected from: a color, an identifier, defects, or non-uniformity.

5. The pharmaceutical apparatus of claim 2, further comprising a programmable controller configured to:
operate the tablet press;
operate the analytical and quality control table;
receive data from the at least one PAT sensor;
receive the image from the imaging device; and
the controller determining at least one of: the potency, water content, impurities, or a presence of polymorphs of the first powdered ingredient and the second powered ingredient from the data collected by the at least one PAT sensor.

6. The pharmaceutical apparatus of claim 1, wherein the analytical and quality control table further comprises a labeler configured to mark a pharmaceutical tablet with a tablet identifier.

7. The pharmaceutical apparatus of claim 6, wherein the labeler comprises an inkjet printer configured to print the tablet identifier on an outer surface of the pharmaceutical tablet.

8. The pharmaceutical apparatus of claim 1, further comprising:
a weighing scale configured to operate with a standard deviation between 0 and 3; and
weight information obtained from the scale being used in combination with size data processed by the controller to determine density of each layer of the pharmaceutical tablet.

9. The pharmaceutical apparatus of claim 8, further comprising an imaging device and a labeler.

10. The pharmaceutical apparatus of claim 1, wherein the first powdered ingredient comprises an active pharmaceutical ingredient.

11. The pharmaceutical apparatus of claim 1, wherein the first powdered ingredient comprises an inactive ingredient.

12. The pharmaceutical apparatus of claim 1, further comprising a discharge chute configured to transfer the pharmaceutical tablet from the tablet press to the analytical and quality control table.

13. The pharmaceutical apparatus of claim 1, wherein the PAT sensor is located downstream of the tablet press and a tablet discharge chute, further comprising a programmable controller configured to:
operate the tablet press;
operate the analytical and quality control table; and
receive data from the at least one PAT sensor.

14. A pharmaceutical apparatus comprising:
a tablet press, including a die and a punch;
a first dosing unit configured to insert a first powdered ingredient into the die of the tablet press;
a second dosing unit configured to insert a second powdered ingredient into the die of the tablet press; and
an analytical and quality control table including a transfer mechanism and a sensor;
wherein the punch of the tablet press is configured to compress at least one powdered ingredient in the die into at least a first layer of a pharmaceutical tablet,
wherein the transfer mechanism is configured to transfer the pharmaceutical tablet to a position accessible by the sensor;
wherein when the analytical and quality control table includes the sensor which is configured to assist in determining at least one of: hardness, water content, impurities, or a presence of polymorphs of the pharmaceutical tablet.

15. The pharmaceutical apparatus of claim 14, wherein the analytical and quality control table further comprises a labeler configured to mark a pharmaceutical tablet with a tablet identifier.

16. The pharmaceutical apparatus of claim 15, wherein the labeler comprises an inkjet printer configured to print the tablet identifier on an outer surface of the pharmaceutical tablet.

17. The pharmaceutical apparatus of claim 14, wherein the first powdered ingredient comprises an active pharmaceutical ingredient.

18. The pharmaceutical apparatus of claim 14, wherein the first powdered ingredient comprises an inactive ingredient.

19. The pharmaceutical apparatus of claim 14, wherein the tablet press further comprises a discharge chute configured to transfer the pharmaceutical tablet from the tablet press to the analytical and quality control table.

20. The pharmaceutical apparatus of claim 14, further comprising a camera capturing an image of each of the layers of the pharmaceutical tablet to assist in determining a hardness of the pharmaceutical tablet.

21. The pharmaceutical apparatus of claim 14, further comprising a camera capturing an image of each of the layers of the pharmaceutical tablet to assist in determining at least one characteristic of each layer of the pharmaceutical tablet, the at least one characteristic selected from: thickness, color, identifier, or non-uniformity.

22. The pharmaceutical apparatus of claim 14, wherein:
the sensor comprises at least one of: an ultraviolet-visible spectrometer, a near-infrared spectrometer, a Raman spectrometer, a fluorescence spectrometer, a refractometer, or a micro-indentation sensor;
further comprising a programmable controller configured to:
operate the tablet press;
operate the analytical and quality control table;
receive data from the sensor when the analytical and quality control table includes the sensor; and
receive the image from the imaging device when the analytical and quality control table includes the imaging device.

23. The pharmaceutical apparatus of claim 14, wherein:
the analytical and quality control table further comprises a weighing scale configured to operate with a standard deviation between 0 and 3; and
weight information obtained from the scale being used in combination with size data processed by a programmable controller to determine density of each layer of the pharmaceutical tablet.

24. The pharmaceutical apparatus of claim 14, further comprising a tablet printer.

25. The pharmaceutical apparatus of claim 14, wherein the sensor comprises a pre-pressing PAT sensor configured to assist in determining potency of any of the first powdered ingredient or the second powdered ingredient as the first powdered ingredient flows into the first dosing unit or as the second powdered ingredient flows into the second dosing unit.

26. A method of manufacturing a pharmaceutical tablet, the method comprising:
(a) receiving details of the pharmaceutical tablet order for a number of tablets at a programmable controller, the details corresponding to at least one of: a color of the first layer of the pharmaceutical tablet, a color of the second layer of the pharmaceutical tablet, a tablet identifier of the pharmaceutical tablet, or a maximum amount of acceptable visual defects of the pharmaceutical tablets;
(b) controlling operation of a first dosing unit, a second dosing unit, a tablet press, an analytical and quality control platform, a sensor and an imaging camera, with the controller;
(c) automatically determining an amount of a first powdered ingredient to be included in the pharmaceutical tablet and an amount of a second powdered ingredient to be included in the pharmaceutical tablet with the controller based at least in part on the details;
(d) sending a signal from the controller to the first dosing unit directing the first dosing unit to insert the amount of the first powdered ingredient into a die of the tablet press;
(e) sending a signal from the controller to the tablet press directing the tablet press to extend a punch to compress the first powdered ingredient to form the first layer of the pharmaceutical tablet;
(f) sending a signal from the controller to the second dosing unit directing the second dosing unit to insert the amount of the second powdered ingredient into the die of the tablet press;
(g) sending a signal from the controller to the tablet press directing the tablet press to extend the punch to compress the second powdered ingredient to form the second layer of the pharmaceutical tablet;
(h) sending a signal from the controller to the tablet press directing the tablet press to eject the pharmaceutical tablet from the tablet press;
(i) transferring the pharmaceutical tablet to the quality control platform;
(j) sending a signal from the controller to the quality control platform directing the quality control platform to place the pharmaceutical tablet in a position wherein the pharmaceutical tablet is accessible by the sensor and the camera;
(k) sending a signal from the controller to the sensor and the camera to gather data related to the pharmaceutical tablet; and
(l) sending the data related to the pharmaceutical tablet from the sensor and the camera to the controller, the data assisting the controller in determining at least one of: potency, water content, impurities, or a presence of polymorphs of the first powdered ingredient and the second powered ingredient.

27. The method of claim 26, wherein the tablet press ejects the pharmaceutical tablet to a discharge chute configured to transfer the pharmaceutical tablet to the quality control platform.

28. The method of claim 26, the method further comprising:
determining whether the data related to the pharmaceutical tablet satisfies the details of the pharmaceutical tablet order by the controller;
wherein when the data does not satisfy the details of the pharmaceutical tablet order, the controller sends a signal to the quality control platform directing the quality control platform to reject the pharmaceutical tablet and adjusts the determination of the amount of at least one of: the first powdered ingredient or the second powdered ingredient in step (b); and
wherein when the data satisfies the details of the pharmaceutical tablet order, the controller sends a signal to the quality control platform directing the quality control platform to accept the pharmaceutical tablet.

29. A method of manufacturing a pharmaceutical tablet, the method comprising:
(a) inserting an amount of a first powdered ingredient into a die of a tablet press;
(b) inserting an amount of a second powdered ingredient into the die of the tablet press;
(c) compressing the first powdered ingredient and the second powdered ingredient into the pharmaceutical tablet;
(d) transferring the pharmaceutical tablet to an analytical and quality control station which is physically separated from the tablet press and comprises at least one of: an ultraviolet-visible spectrometer sensor, a near-infrared spectrometer sensor, a Raman spectrometer sensor, a fluorescence spectrometer sensor, a refractometer sensor, or a micro-indentation sensor; and
(e) using software, stored in non-transient memory, to automatically determine at least one of: potency, hardness, water content, impurities, or a presence of polymorphs of the pharmaceutical tablet, with the assistance of a signal from the sensor.

30. The method of manufacturing a pharmaceutical tablet of claim 29, wherein the analytical and quality control station further comprises a camera capturing a dimension of each layer of the pharmaceutical tablet.

31. The method of manufacturing a pharmaceutical tablet of claim 29, wherein the method further comprises determining a hardness of the pharmaceutical tablet, wherein an image from a camera assists in determining the hardness of the pharmaceutical tablet.

32. The method of manufacturing a pharmaceutical tablet of claim 29, further comprising determining at least one characteristic of a first layer of the pharmaceutical tablet, the at least one characteristic selected from: a thickness, a color, an identifier, or a non-uniformity, with assistance of an image from the camera.

33. The method of manufacturing a pharmaceutical tablet of claim 32, further comprising determining at least one characteristic of a second layer of the pharmaceutical tablet, the at least one characteristic selected from: a thickness, a color, an identifier, or a non-uniformity, with assistance of the image from the camera.

34. The method of manufacturing a pharmaceutical tablet of claim 29, further comprising compressing the first powdered ingredient into a first layer of a pharmaceutical tablet before inserting the amount of the second powdered ingredient into the die of the tablet press.

35. The method of manufacturing a pharmaceutical tablet of claim 29, further comprising determining at least one of the water content, the impurities, or the presence of the polymorphs of any of the first powdered ingredient or the second powdered ingredient as the first powdered ingredient flows into the first dosing unit or as the second powdered ingredient flows into the second dosing unit by the at least one sensor which includes a pre-pressing PAT sensor.
